(19) United States
(12) Patent Application Publication
(10) Pub. No.: US 2004/0147868 A1
(43) Pub. Date: Jul. 29, 2004

(54) Myocardial implant with collar

(76) Inventors: Earl Bardsley, Newton, MA (US); Peter Boekstegers, Diessen (DE); Patrick Cahalan, Bedford, NH (US); Michael Igoe, Salem, NH (US); Jianlu Ma, Merrimack, NH (US); Anna McVittie, Somerville, MA (US); Laurence Roth, Windham, NH (US)

Correspondence Address: Susanne T. Jones, FINNEGAN, HENDERSON, FARABOW,, GARRETT & DUNNER, L.L.P., 1300 I Street, N.W., Washington, DC, 20005-3315, US

(21) Appl. No.: 10/351,438

(22) Filed: Jan. 27, 2003

(51) Int. Cl. A61M 5/00

(52) U.S. Cl. 604/8

(57) ABSTRACT

A device for treating a heart includes an implant and a collar. The implant may be configured to be positioned in a heart wall between a coronary vessel and a chamber of the heart. The collar may be configured to be disposed externally on a portion of the implant such that the collar extends from the implant so as to contact the heart wall for anchoring the implant in position in the heart wall.

### FIELD OF THE INVENTION

The present invention relates generally to a medical implant for inserting into body tissue and a method of delivering a medical implant. The present invention may also relate to an implant configured to provide flow communication between blood-containing coronary structures, such as, for example, between two coronary vessels or between a heart chamber and a coronary vessel.

### BACKGROUND

An implant for insertion into body tissue may have various uses, such as providing flow communication between two body parts, delivering drugs into a body part, or serving as a sensor, controller, or monitoring device, for example. Without limiting the scope of the present invention, the following paragraphs describe an exemplary use of an implant, such as a stent or conduit, for example, to treat blockages in coronary vessels. The examples discussed below do not constitute a limitation on the scope and applications of the present invention.

Coronary artery disease may be treated with several approaches. Coronary arteries, as well as other coronary vessels, frequently become clogged with plaque which, at the very least, can reduce blood and oxygen flow to the heart muscle (myocardium). The plaque also may impair the efficiency of a heart's pumping action and lead to heart attack or death. In some cases, these coronary arteries can be unblocked through noninvasive techniques, such as, for example, performing balloon angioplasty or stenting a vessel to provide a blood passageway. In more difficult cases, performing a surgical bypass of the blocked vessel may be necessary.

One conventional treatment for a clogged coronary artery is a coronary bypass operation wherein one or more venous segments are inserted between the aorta and the coronary artery. The inserted venous segments or transplants bypass the clogged portion of the coronary artery and thus provide a free and unobstructed flow communication of blood between the coronary artery and the heart. Such conventional coronary artery bypass surgery, however, may be expensive, time-consuming, and traumatic to a patient. Hospital stay subsequent to surgery and convalescence generally is prolonged. Furthermore, many patients may not be suitable surgical candidates due to other concomitant illnesses.

An alternative to coronary artery bypass, angioplasty, and vessel stenting includes providing a flow passage in the myocardial wall between the left ventricle and the coronary artery. The passage may be provided at a point downstream of the blockage. In this technique, a portion of the blood from the left ventricle flows directly through the passage in the myocardial wall and into the artery downstream of the blockage. A variation of this technique includes placing a stent in the heart wall to provide the blood flow passage between the left ventricle and coronary artery.

FIG. 1 illustrates a partial cross-sectional view of a heart 10 having an implant in the form of a stent 12 disposed in a heart wall MYO. As shown, the stent 12 extends between a left ventricle LV and a coronary artery CA. The coronary artery CA has a posterior wall 14 and an anterior wall 16. The stent 12 is positioned at a point in the coronary artery CA downstream of an occlusion or blockage BL of the coronary artery CA. In general, the occlusion or blockage BL in the coronary artery CA can be partial or full so as to inhibit or completely block the blood flow through the coronary artery CA. When used herein, the terms “occlusion” and “blockage” are intended to include full and partial occlusions or blockages. For the stent 12 positioned between the left ventricle LV and coronary artery CA, connecting positions other than the position depicted in FIG. 1 can be utilized. For example, the stent 12 may form a vertical or angled position with respect to the posterior wall 14 of the coronary artery CA or the side of the left ventricle LV. The connection position may be selected so as to avoid interference with various structures in the heart, including the papillary muscles, chordae, and mitral valve, for example.

A problem that may be encountered when using a stent or other type of implant is migration. Migration of the stent after its insertion may lead to the protrusion of the stent beyond the heart wall, for example, either into the left ventricle or into the blood flow lumen of the coronary artery. Migration may create a risk that the stent may not extend completely through the heart wall to establish an unobstructed passageway for blood flow. Migration may also allow portions of the myocardial tissue that surround a passageway to advance toward the passageway and cause the passageway to contract, especially when the myocardial tissue is not sufficiently supported by the stent. The contraction of the passageway may reduce or entirely block blood flow through the stent, thereby rendering the stent less effective in providing an unblocked channel of blood flow to the artery. In addition to the reduction or complete blockage of blood flow, migration of the stent from a designated location potentially will interfere with other structures in the heart and blood vessels and may pose serious risk.

### SUMMARY OF THE INVENTION

Some advantages and purposes of the invention will be set forth in part in the description which follows, and may be obvious from the description, or may be learned by practice of the invention. It should be understood that skilled artisans may practice the invention without having one or more features of any of the objects, aspects, or embodiments described herein. In addition, such features are exemplary and at least some of them are set forth in the detailed description which follows.

An exemplary aspect of the invention includes a device for treating a heart. The device includes an implant and a collar. The implant is positioned in a heart wall between a coronary vessel and a chamber of the heart. The collar is configured to be disposed externally on a portion of the implant such that the collar extends from the implant so as to contact the heart wall for anchoring the implant in position in the heart wall.

Another exemplary aspect of the invention includes a method of treating a heart. The method includes providing a collar and an implant, delivering the collar and the implant to a location proximate to a heart wall, and inserting the collar and the implant into a heart wall between a coronary vessel and a heart chamber. The collar is configured to be disposed externally on a portion of the implant. Also, the collar is configured to extend from the implant so as to contact the heart wall for anchoring the implant in position in the heart wall.

### BRIEF DESCRIPTION OF THE DRAWINGS

The accompanying drawings, which are incorporated in and constitute a part of this specification, illustrate exemplary embodiments of the invention. Those embodiments, together with the following description, serve to explain certain principles and provide a further understanding of the invention. In the drawings,

FIG. 1 is a cross-sectional view of a heart with a stent disposed in the heart wall between the left ventricle and coronary artery downstream of an occlusion in the coronary artery;

FIG. 2 is a cross-sectional view of a heart with an exemplary embodiment of a collared stent disposed in the heart wall between the left ventricle and coronary artery downstream of an occlusion according to an aspect of the invention;

FIG. 3A is a perspective view of an exemplary embodiment of a stent with a collar according to an aspect of the present invention;

FIG. 3B is a partial, cross-sectional view of a heart implanted with the collared stent of FIG. 3A according to an exemplary aspect of the invention;

FIG. 4A is a perspective view of another exemplary embodiment of a stent with a collar according to an aspect of the present invention;

FIG. 4B is a partial, front view of the collar of FIG. 4A in an unwrapped position according to an aspect the present invention;

FIG. 4C is a perspective view of yet another exemplary embodiment of a collar according to an aspect of the present invention;

FIG. 4D is a partial, cross-sectional view of a heart shown implanted with the collared stent of FIG. 4A according to an aspect of the invention;

FIG. 5A is a perspective view of another exemplary embodiment of a stent with a collar according to an aspect of the present invention;

FIG. 5B is a front view of an exemplary embodiment of a coil structure used to form a collar according to an aspect of the present invention;

FIG. 5C is a front view of another exemplary embodiment of a coil structure used to form a collar according to an aspect of the present invention;

FIG. 5D is an enlarged front view of the area D in FIG. 5B according to an aspect of the present invention;

FIG. 5E is a partial, cross-sectional view of a heart shown implanted with the collared stent of FIG. 5A according to an exemplary aspect of the invention;

FIG. 6 is a side view of an exemplary embodiment of a portion of a coil type stent according to an exemplary aspect of the present invention.

FIG. 7 is a perspective view of a collar welded to a stent according to an exemplary aspect of the present invention;

FIG. 8 is a partial, cross-sectional view of a heart shown implanted with an exemplary embodiment of a collared stent having extensions according to an aspect of the invention;

FIG. 9 is a cross-sectional view illustrating an exemplary embodiment of percutaneously inserting a catheter according to an aspect of the invention;

FIG. 10 is a cross-sectional view illustrating another exemplary embodiment of percutaneously inserting a catheter according to an aspect of the invention;

FIG. 11 is a partial, cross-sectional view of a heart with a catheter inserted in the heart wall according to an exemplary aspect of the invention;

FIG. 12 is a partial, cross-sectional view of a heart with an exemplary embodiment of a collared implant and catheter in a heart wall according to an aspect of the invention;

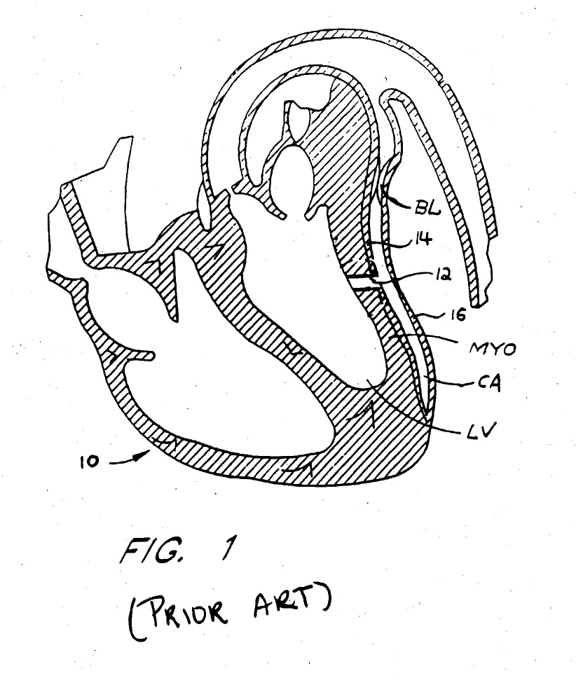

FIG. 1 3A is a partial, cross-sectional view of a heart shown with an exemplary embodiment of a catheter carrying an implant and collar to the heart wall according to an aspect of the invention;

FIG. 13B is a partial, cross-sectional view of a heart shown with another exemplary embodiment of a catheter carrying an implant and collar to the heart wall according to an aspect of the invention; and

FIG. 14 is a partial, cross-sectional view of a heart shown with an exemplary embodiment of a catheter delivering a collar and implant in the heart wall according to an aspect of the invention.

### DESCRIPTION OF EXEMPLARY EMBODIMENTS

Reference will now be made in detail to exemplary embodiments illustrated in the accompanying drawings. Wherever possible, the same reference numbers will be used throughout the drawings to refer to the same or like parts.

The present invention provides an implant with a collar for anchoring the implant in place in a body part, for example, in body tissue. An implant inserted into body tissue may have various uses, such as, for example, providing flow communication between two body parts, delivering drugs into a body part, or serving as a sensor, controller, or monitoring device. The present invention also provides a heart wall implant, such as a conduit or a stent, for example, with a collar for anchoring the implant in place. In addition, the present invention provides a method of inserting a collared implant.

In one exemplary embodiment described in the following paragraphs, a heart wall implant may provide a direct blood flow passageway between a chamber of a heart, such as the left ventricle, and a coronary vessel, such as a left anterior descending coronary artery. The principles and applications of the present invention, however, are not limited to conduits placed so as to provide direct blood flow between the left ventricle and the coronary artery. Rather, the device and method of the present invention encompass the use of an implant for flow communication from any space within a patient's body to another space within the patient's body, such as between any heart chamber and any blood vessel, between two blood vessels, or between other body spaces. In addition, the device and method also encompass the use of an implant for delivering drugs into a body part or for serving as a sensor, controller, or monitoring device within the body.

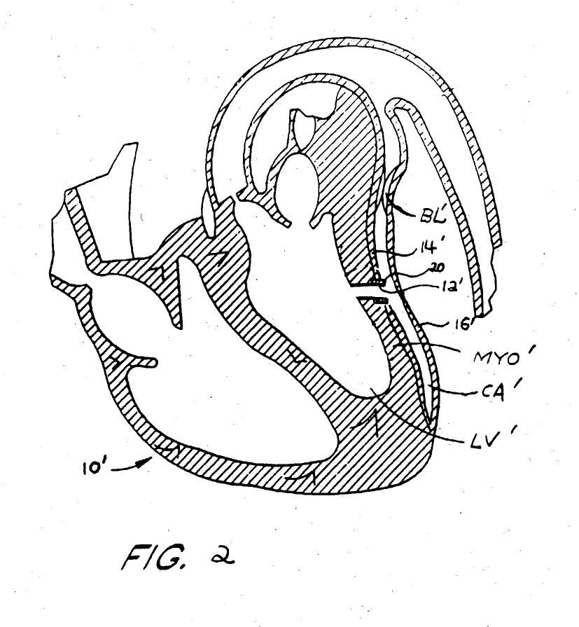

As shown in FIG. 2, a heart 10′ has an implant in the form of a conduit or stent 12′ in a heart wall MYO′. The stent 12′ may extend from proximate the posterior wall 14′ of the coronary artery CA′ to proximate the inner wall of the left ventricle LV′. In other words, the stent 12′ may lie in the heart wall MYO′ such that it is approximately flush with the floor (i.e., the posterior wall 14′) of the coronary artery CA′. Furthermore, the stent 12′ has a collar 20 disposed externally on a portion of the stent 12′ to anchor the stent 12′ in place in the heart wall MYO′.

As discussed above, negative effects may result if the stent 12′ protrudes into the coronary artery CA′ or is recessed within the heart wall MYO′. For example, if the stent 12′ protrudes too far into the lumen of the coronary artery CA′, blood flow through the coronary artery CA′, as well as blood flow exiting from the stent 12′ may become disturbed, resulting in stasis. On the other hand, if the stent 12′ is recessed within the heart wall MYO′ such that a space remains between the stent 12′ and the posterior wall 14′ of the coronary artery CA′, the space may become occluded with heart tissue, thereby hindering or preventing blood flow through the stent 12′ and into the coronary artery CA′.

To enable substantially unencumbered blood flow through an implant, such as in the form of the stent 12′, for example, the implant should be placed and maintained in a designated position. However, in certain applications, it may be desirable to cover the inside, outside, or both, of a heart wall implant with a polymer covering, such as, for example, an expanded polytetrafluoroethylene (ePTFE). Such covering helps to prevent the myocardial tissue from moving into the lumen of the implant when a mesh-type stent is used. But this covering may reduce the frictional force that holds the implant in place and allow the implant to move away from an installed, desired position. In the case of a heart wall implant, the continuous, repetitive heart pumping action and variations in the blood flow also may cause the implant in a heart wall to migrate from its designated location toward a left ventricle or a coronary artery. Thus, the heart wall implant may be vulnerable to migration for various different reasons, especially migration along the axis of the passageway, for example. As explained above, the migration of the implant may create undesirable risks, including but not limited to, stasis, occlusion of the passageway provided by the implant, arterial occlusion, and interference with the functioning of other body components, such as heart structures, for example.

The present invention may eliminate the above-mentioned risks by providing the implant with a collar, such as that shown in FIG. 2. The collar may help to anchor the implant, which may be in the form of a conduit or stent, in place in the heart wall so as to help preventing migration of the implant.

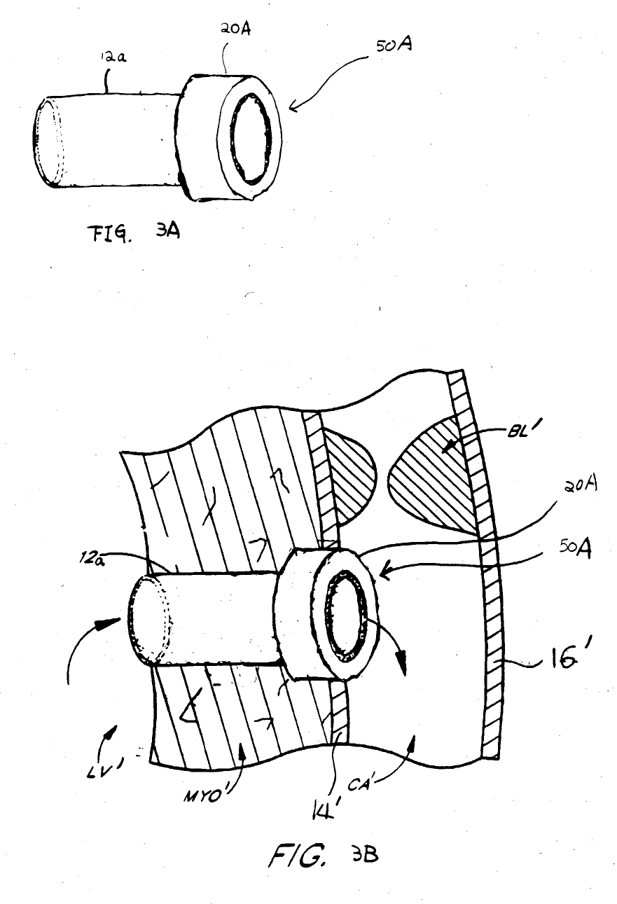

FIG. 3A shows an exemplary embodiment of a collared stent 50A according to an aspect of the invention. A collar 20A is provided externally on a portion of the stent 12a. The length of the collar 20A is less than that of the stent 12a along the axial direction. The collar 20A may have a slightly larger outer diameter than the stent 12a and the collar 20A may have an inner diameter that is approximately equal to the outer diameter of the stent 12a. The collar 20A is substantially ring-shaped and circularly surrounds a portion of the exterior wall of the stent 12a. The collar 20A may have grooves, lines, edges, ribs, teeth, or other similar features (not shown in FIG. 3A) on its inner, outer, or both surfaces to further enhance its attachment to the stent 12a and/or surrounding tissue. Although FIG. 3A illustrates an example in which the collar 20A sits on an end portion of the stent 12a, the collar 20A may be placed anywhere along the length or external wall of the stent 12a.

The stent 12a in this exemplary embodiment may be a non-mesh stent, a mesh stent, a coil type stent, or other type of stent, for example. Some exemplary embodiments of suitable coil type stents are provided in U.S. application Ser. No. 09/917,655, entitled “Myocardial Stents and Related Methods of Providing Direct Blood Flow from a Heart Chamber to a Coronary Vessel,” filed Jul. 31, 2001, the entire disclosure of which is incorporated by reference herein. Also, stent 12a may be provided with a covering. FIGS. 3A and 3B show an example of a stent having a smooth surface, such as a covered mesh stent, a covered coil-type stent, or a non-mesh stent, for example. When a covered stent is used, the covering may be in the form of a polymer material, for example, an ePTFE. The stent 12a may have a covering on the external surface, inner surface, or both.

The collar 20A and/or stent 12a may be elastic or have expandable and/or collapsible structures. An elastic, expandable, and/or collapsible collar may be crimped onto and joined with a collapsed stent. The combination of the collapsed stent and collar may facilitate the delivery of the combination into the heart wall MYO′ by providing a smaller structure during delivery.

The collar 20A and/or the stent 12a can be made from a biocompatible metal material, such as, for example, stainless steel, nickel (Ni) alloys, titanium (Ti) alloys, nickel-titanium alloys, cobalt-based alloys, titanium, tantalum, and other similar suitable metal materials. Examples of nickel, titanium, or nickel-titanium alloys may include NiTi shape memory alloys and NiTi super elastic alloys. Alternatively, the collar 20A and/or the stent 12a can be made from a biocompatible polymer. In particular, a material that provides adequate friction against the surface of the stent 12a and heart wall MYO′ or other body tissues may be a desirable material for the collar 20A. Examples of biocompatible polymers include polytetrafluoroethylenes (PTFEs), polyetheretherketones (PEEKs), polyesters, polyurethanes, polyamides, ePTFEs, and other similar suitable polymers.

When implanted in the heart wall, the collar 20A may provide an anchoring effect to hold the stent 12a in place. The collar 20A radially extends from the stent 12a so as to contact the surrounding heart wall MYO′. The collar 20A may grab onto the surrounding myocardial tissue in contact with its outer surface by its structural extension into the heart wall MYO′, by friction provided by its material, or by both, for example. Similarly, the collar 20A may also grab onto the stent 12a in contact with its inner surface by its structure, for example, by friction. Alternatively, the collar 20A may be connected to the stent 12a by a welding connection, by a sewing connection, by some other connection means, or by a combination of two or more connection means. The attachment of the collar 20A to both the stent 12a and the heart wall MYO′ helps to prevent or hinder the migration of the stent 12a.

FIG. 3B shows the collared stent 50A implanted in the heart wall MYO′ between the left ventricle LV′ and the coronary artery CA′. As shown in FIG. 3B, the collar 20A extends radially from the stent 12a into surrounding myocardial tissue. It may also extend into portions of the surrounding posterior wall 14′ of the coronary artery CA′. Its extension into the heart wall MYO′ and also possibly portions of the posterior wall 14′ allows the collar 20A to provide additional frictional force, for example, above and beyond the frictional force that acts on a stent without a collar, to hold the stent 12a in place. In this example, the collar 20A is provided at an end portion of the stent 12a, for example, the end proximate the coronary artery CA′. In one embodiment, placing the collar 20A at or near the coronary artery CA′ may help to maintain the position of the stent 12a relative to the coronary artery CA′ so as to maintain blood flow through the stent 12a and into the coronary artery. It should be noted, however, that the location of the collar 20A is not limited to this position and can be anywhere along the length of the stent 12a.

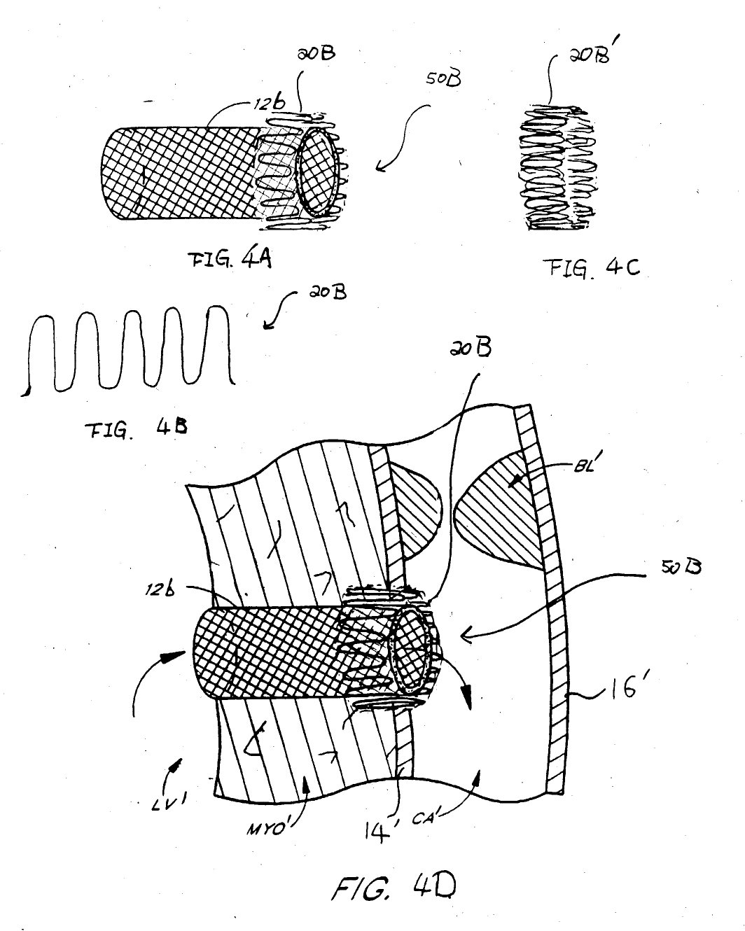

FIG. 4A shows another exemplary embodiment of a collared stent 50B.

In this example, a coil collar 20B circularly surrounds an external portion of a mesh stent 12b to provide an external coil structure. Similar to the collar 20A described above, the outer diameter of the collar 20B may be larger than the outer diameter of the stent 12b when placed on the stent 12b, and the length of the collar 20B is less than that of the stent 12b along the axial direction. Similarly, the collar 20B may sit anywhere along the length or external wall of the stent 12b, such as, for example, on an end portion of the stent 12b proximate the coronary artery CA′.

Although FIGS. 4A and 4D show an example of a mesh stent, the stent 12b may be a covered mesh stent, a covered coil-type stent, or a non-mesh stent that provides a covering on the external surface, inner surface, or both, as discussed with reference to the stent 12a of FIGS. 3A and 3B.

The coil collar 20B may be formed by a thin coil that wraps around the stent 12b in a repeating, wave-like form, as shown in FIG. 4B, which depicts the collar 20B unwound and laid flat. As shown, the coil may have a sinusoidal wave-like form. Aside from the repeating, wave-like pattern shown in FIGS. 4A, 4B, and 4D, the coil may have a random pattern while wrapping around the stent 12b. Both the wave-like and random patterns may enable the coil collar 20B to be crimped and expanded freely during the delivery and installation of the coil collar 20B. The patterns also may enable the coil collar 20B to grab onto the myocardial tissue of the heart wall MYO′ and the stent 12b. Those skilled in the art would recognize various patterns the coil collar could have so as to expand freely during implantation. For example, a coil collar could have a structure similar to those used for coil type stents.

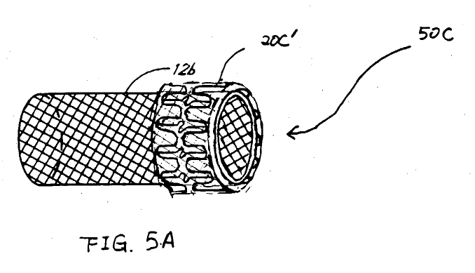

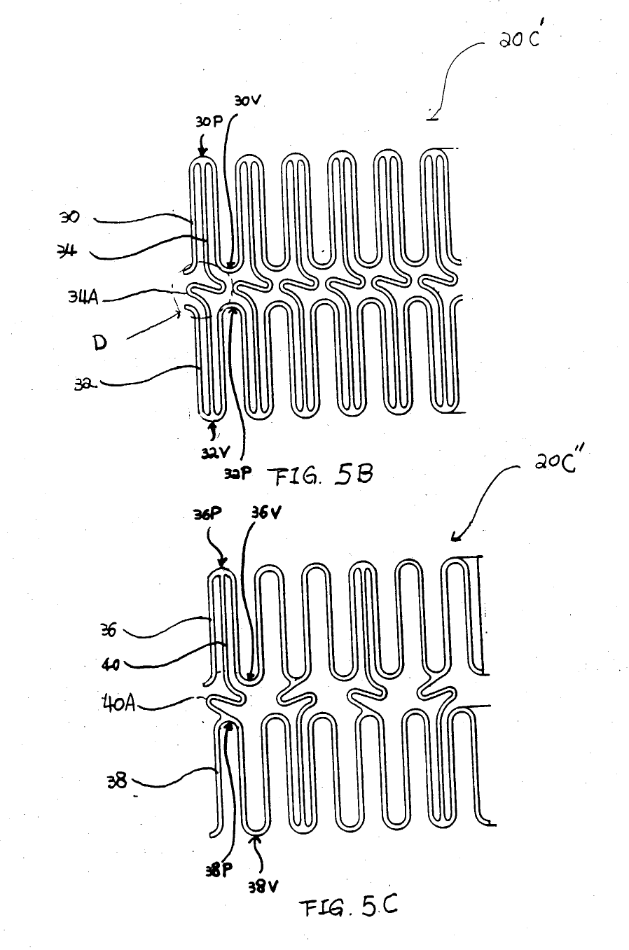

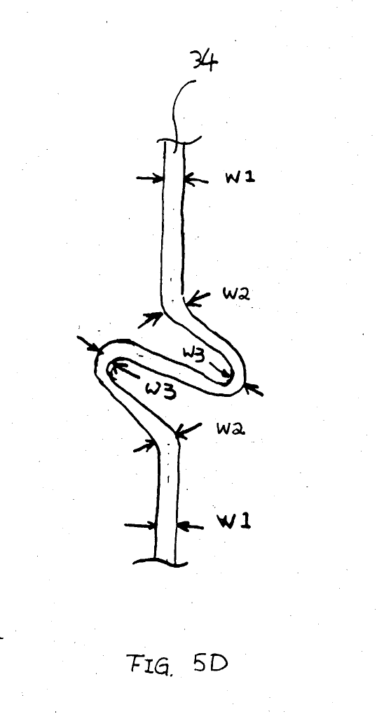

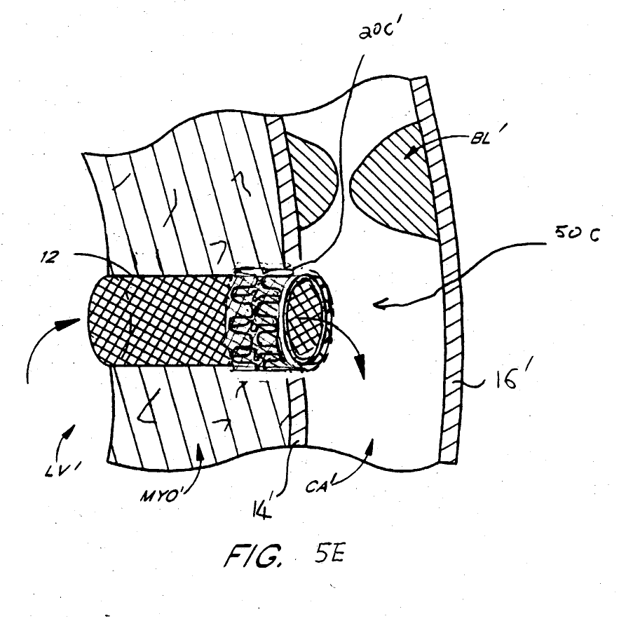

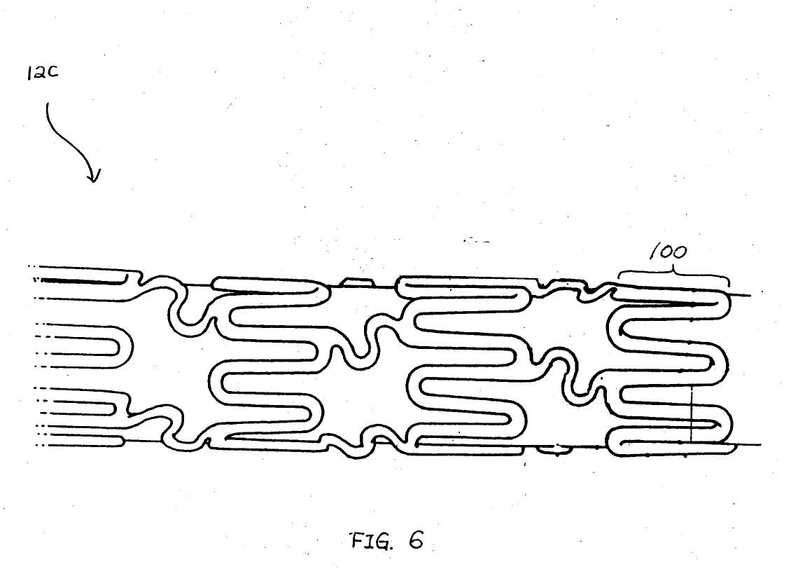

In another exemplary embodiment shown in FIG. 4C, a coil collar 20B′ may include multiple coils layered over one another. The layers of coils provide a denser coil arrangement that may enhance the collar's frictional effect and strength. Also, in an exemplary embodiment (not shown), the collar may be a mesh collar that provides similar effects as a coil collar. The mesh collar may be formed with the material for forming a mesh stent or a coil type stent or with the materials provided below for forming coil collars. In addition, rather than the mesh stent 12b, a stent having a coil structure similar to those in FIGS. 4A, 4C, 5B, and 5C, or other suitable coil structures could be employed with any of the collars described herein.

The coil collars described in FIGS. 4A-4C can be made from a biocompatible metal discussed above for the collar 20A in FIGS. 3A and 3B.

Alternatively, the coil collars can be made from a biocompatible polymer discussed above for the collar 20A.

In the aforementioned embodiments shown in FIGS. 4A-4C, the various collars may serve as a structure that extends from an external portion of the implant so as to contact surrounding tissue. The collar may grab onto both the surrounding tissue outside it and the implant inside it by its extension into the body tissue, its material, and/or its structure. Accordingly, the collar may help to restrain the migration of the implant.

Referring to FIG. 4D, the coil collar 20B extends into the surrounding tissue of the heart wall MYO′ when the stent 12b with the coil collar 20B is inserted in the heart wall MYO′. In one embodiment, the coil collar 20B may be pressed toward the surrounding myocardial tissue when inserted into and expanded in a passageway provided within the heart wall MYO′. When pressed toward the surrounding tissue, the open, wire-like segment structure of the coil collar 20B may permit it to be substantially surrounded or partially buried by the surrounding tissue. Accordingly, the partial enclosure of the coil by the tissue may help the heart wall MYO′ to hold the coil collar 20B in place. In addition, the surrounding tissue also may exert force toward the stent 12b through the collar 20B, as a result of the stent and collar insertion and expansion in this example. That force creates additional frictional force between the stent 12b and the collar 20B to hold the stent 12b in place. In this way, the frictional force further helps to prevent the stent 12b from migrating. The open structure of a mesh collar may permit similar effects.

FIG. 5A shows yet another exemplary embodiment of a collared stent 50C that has a coil collar 20C′ surrounding an external portion of the stent 12b. The coil collar embodiments exemplified in FIGS. 5A-5E may provide similar functions as the coil collars in FIGS. 3A-3C, but may have different arrangements of coil or coils. These coil collars provide structural flexibilities, such as, for example, flexibilities along both axial and radial directions. As will be explained, the coil collars of FIGS. 5A-5E may be implemented with various forms and patterns in their coil design.

Referring to FIG. 5B, an exemplary design of a coil collar 20C′ provides two coils with interconnecting members between them. The upper coil 30 wraps around an implant (e.g., the stent 12b) in a sinusoidal, or other suitable wave-like form. Similarly, the lower coil 32 wraps around an implant (e.g., the stent 12b) in a sinusoidal, or wave-like form. In this example, the lower coil 32 is placed in a mirrored position relative to the upper coil 30. That is, each peak of the upper coil 30, such as the peak 30P, is vertically aligned with each valley of the lower coil 32, such as the valley 32V. Similarly, each valley of the upper coil 30, such as the valley 30V, is vertically aligned with each peak of the lower coil 32, such as the peak 32P. Although the upper coil 30 and lower coil 32 shown in FIG. 5B have the same waveform, they may have different waveforms in other embodiments.

FIG. 5B shows an exemplary embodiment of using one or more interconnecting members to connect the upper coil 30 to the lower coil 32. In one embodiment, an interconnecting member comprises a vertical strut 34 connecting the peak 30P of the upper coil 30 to the corresponding valley 32V of the lower coil 32, for example. In this embodiment, multiple vertical struts may be used to connect other peaks of the upper coil 30 to the corresponding valleys of the lower coil 32 in the same way, as shown in FIG. 5B. The vertical strut 34 may have a flexible joint 34A proximate the middle of the strut to offer flexibility in extending, contracting, and bending. As an example, the flexible joint 34A may include an “S” or “Z” shape articulation joint. The flexible joint 34A and other similar joints illustrated in FIG. 5B merely serve as illustrative examples. An interconnecting member for the collar 20C′, therefore, may incorporate any type of twist, curve, bend, or similar structure to provide the collar 20C′ with structural flexibility or elasticity without departing from the scope of the invention. In this example, flexibility may be provided along the axial direction of the coil collar 20C′ to allow extension, contraction, and/or bending thereof. Also, flexibility may be provided along other directions, including transverse and radial directions, which may permit bending of the coil collar 20C′ and may permit the upper coil 30 and the lower coil 32 to shift from side to side relatively to each other or to move relatively to each other in any other directions.

FIG. 5C shows an alternative exemplary design of a coil collar 20C″ that provides two coils with one or more interconnecting members between them. The coil collar 20C″ has an upper coil 36 and a lower coil 38 that are similar to the upper and lower coils of the coil collar 20C′ in FIG. 5B. But the location of the upper coil 36 relative to the lower coil 38 differs than that of coil collar 20C′ . That is, each peak of the upper coil 36, such as the peak 36P, is vertically aligned with each peak of the lower coil 38, such as the peak 38P. Also, each valley of the upper coil 36, such as the valley 36V, is vertically aligned with each valley of the lower coil 38, such as the valley 38V. Although FIGS. 5B and 5C provide two examples of the upper and lower coil alignment, skilled artisans would understand that the sizes, axial lengths, thicknesses, wave lengths, coil patterns, and relative locations of the upper and lower coils may vary without being limited by these two examples.

FIG. 5C shows another exemplary embodiment of using one or more interconnecting members to connect the upper coil 36 to the lower coil 38. In one embodiment, an interconnecting member comprises a vertical strut 40 connecting the peak 36P of the upper coil 36 to the corresponding peak 38P of the lower coil 38, for example. Similar to the example illustrated in FIG. 5B, multiple vertical struts may be used to connect other peaks of the upper coil 36 to the corresponding peaks of the lower coil 38, as shown in FIG. 5C. Similarly, the vertical strut 40 has a flexible joint 40A that includes an “S” or “Z” shape articulation joint and/or any type of twist, curve, bend, or similar structure. In the exemplary embodiment of FIG. 5C, the joint 40A is positioned proximate an end of the vertical strut 40. The flexible joint 40A provides the collar 20C″ with structural flexibility, including flexibility along the axial direction to allow free extension, contraction, and/or bending. The flexible joint 40A also may provide flexibility in other directions, including transverse and radial directions, which may permit bending of the coil collar 20C″ and may permit the upper coil 36 and the lower coil 38 to shift from side to side relatively to each other or to move relatively to each other in any other directions.

As described above, the flexible joint for an interconnecting member between an upper coil and a lower coil may allow relative movements between the upper coil and the lower coil of FIG. 5B or 5C, such as, for example, allowing the two coils to move toward each other, to move away from each other, and to shift their relative transverse and/or radial positions, for example, when the collar is twisted.

Therefore, the flexible joint shown in FIGS. 5B and 5C may allow a collar to be extended, contracted, and twisted relatively easily.

In one embodiment, the vertical struts 34 and 40 may be made to have smaller widths than the upper and lower coils, 30, 32, 36, and 38 and the design may increase flexibility in the extension, contraction, bending, and/or twisting of coils. Furthermore, the vertical struts 34 and 40 may have smaller width or widths at the twisted or curved portions of their flexible joints then at the straight portions. FIG. 5D shows an enlarged front view of an area D near the flexible joint 34A in FIG. 5B. In FIG. 5D, W2 and W3 represent the widths of the vertical strut 34 at or near its curved portions, and W1 represents the width of the vertical strut 34 at its straight portions. As an example, W2 and W3 may be smaller than W1 so as to allow the flexible joint 34A to be bent more easily and provide more elasticity at the curved portions. In addition, W2 may be equal to W3, although the two may have different values depending on a particular design.

The coils in a coil collar, such as the collars 20C′ and 20C″, may be made from a biocompatible metal discussed above for the collar 20A in FIGS. 3A and 3B. Alternatively, a coil collar can be made from a biocompatible polymer, as discussed above for the collar 20A.

FIGS. 5A-5C depict coil collars having a single layer of coil. However, it is envisioned that a coil collar of the present invention could have multiple coils layered on top of each other in a manner similar to the layered multi-coil structure described with respect to FIG. 4C. The multiple layer structure of the coil collar may further enhance the strength of a coil collar and its ability to grab onto surrounding myocardial tissue. By altering the density of the multiple layers, the strength and ability to grab onto the myocardial tissue may be altered.

Referring to FIG. 5E, when a collared stent 50C is implanted in the heart wall MYO′, the interactions between the coils of the collared stent 50C and surrounding myocardial tissue are similar to those discussed above in FIG. 4D. Accordingly, the coil or coils of the collared stent 50C may be substantially surrounded or partially buried by the surrounding myocardial tissue. The partial enclosure of the coil or coils by the tissue may help the heart wall MYO′ to hold the coil collar 20C′ and the stent 12b in place. However, the coil collar 20C′ may provide more flexibility during delivery and insertion with its structural design.

The coils for the collars 20B, 20B′, 20C′ and 20C″ in FIGS. 4A-4D and 5A-5E may have a square, rectangular, oval, circular, or other suitable cross-section. In other words, the coil can be cut or otherwise formed from a raw material to provide a desirable cross-sectional shape, such as a rectangular cross-sectional shape, for example. The coils may be formed by a laser cutting, chemical etching or electrochemical method, or other suitable method for shaping metal or polymer materials. In addition, rather than manufacturing the components of a coil collar separately, the components of a coil collar may be made as one integral body from the same material. As an example, an upper coil, a lower coil, and one or more interconnecting members may be formed as one integral body in the manufacturing process.

As an exemplary embodiment, the coil collars may be made of a 316L stainless steel and the segments of a coil may have a substantially rectangular section. The segments may have the following dimensions. The thickness, as measured in a direction perpendicular to the page containing FIGS. 5B and 5C, for example, may range from about 0.002 in. to about 0.008 in., and may be about 0.0052 in. +/−0.0005 in. in one example. The width of the wire-like segments forming the upper and lower coils may range from about 0.003 in. to about 0.008 in., and may be about 0.0045 in. +/−0.0005 in. in one example. The width, W1, of the vertical struts of FIGS. 5A-5E may range from about 0.003 in. to about 0.008 in., and may be about 0.004 in. +/−0.0005 in. in one example. As noted above, the vertical struts may have a smaller width at the curved portions. For example, the width W2 may range from about 0.003 in. to about 0.008 in., and the width W3 may range from about 0.002 in. to about 0.006 in. Furthermore, the height from the peak to valley of a coil may range from about 0.02 in. to about 0.2 in., and may be about 0.115 in. +/−0.001 in. in one example. The distance between the upper and lower coils may range from about 0.01 in. to about 0.1 in., and may be about 0.03 in. +/−0.001 in. in one example.

In the exemplary embodiments described above, the length of the collar may range from about 0.02 in. to about 0.4 in. For example, the length may range from about 0.08 in. to about 0.16 in. The collar can have any length within or outside these exemplary ranges, and skilled artisans would understand that different dimensions may be used, depending on the applications and designs of the collars and implants, for example.

FIG. 6 shows an exemplary embodiment of a portion of a stent 12c comprising a plurality of stent cells. As an example, a stent cell may be defined as one of the repeating segments of a stent that extends along the length of the stent and that forms a portion of the stent structure. For example, FIG. 6 shows a stent cell 100. In an exemplary embodiment, a collar may extend along the length defined by one or more stent cells, such as, for example, the stent cell 100 of the stent 12c illustrated in FIG. 6. As an example, the collar may have a length of approximately one to two stent cells. However, the collar may have a length of any number of stent cells, including a length that approximately equals the length of the entire stent.

In addition, the inner diameter of a collar, such as the exemplary embodiments of the collars described above, may approximate the outer diameter of a stent to which the collar attaches when the stent and collar are in place in their operative positions. However, it is noted that the various shapes, dimensions, and materials provided above for collars are exemplary and skilled artisans would understand that different shapes, dimensions, and materials may be used, depending on the applications and designs of the collars and implants, for example.

The connection among an implant, collar, and surrounding myocardial tissue may be based on the frictional forces existing at their respective interfaces.

In one aspect of the present invention, the frictional force between a collar and an implant allows the collar to grab onto the implant, thereby helping to prevent the implant from slipping relative to the collar. In addition, when the collar is installed in place, the frictional force between the myocardial tissue and the collar may help the collar to grab onto the heart wall MYO′. Also, the possible intermeddling between the heart tissue and open collar structure, as discussed above for the various coil collars, for example, may also help the collar to grab onto the heart wall MYO′. Consequently, the collar may anchor the implant in place within the heart wall by relying on the frictional effects.

To further enhance the attachment of a collar to an implant, the collar may be physically affixed to the implant via various means, such as a sewing connection, a welding connection, or other suitable connections, for example. In an exemplary embodiment, a collar may be sewn to an implant. For, example, a collar may be sewn to an implant with threads, thin wires, or sutures to attach the collar to the implant. The threads, thin wires, or sutures may be made of a biocompatible material, for example. In an exemplary embodiment, the sewing connection between the collar and the implant may help to maintain the relative positions between the two and may help to anchor the implant in place in a body part.

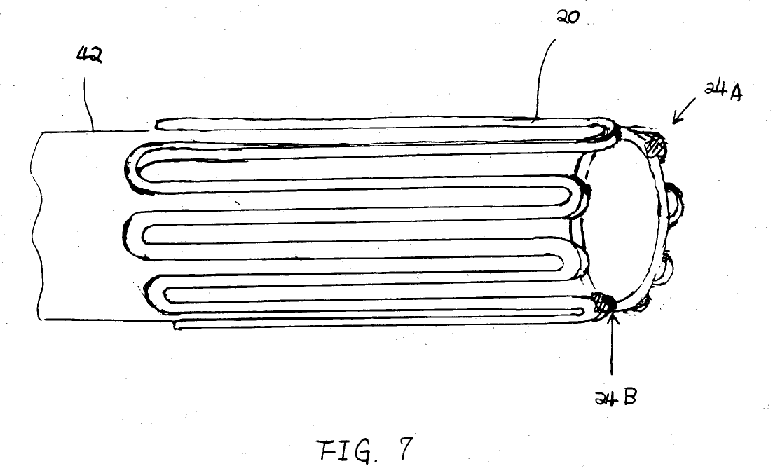

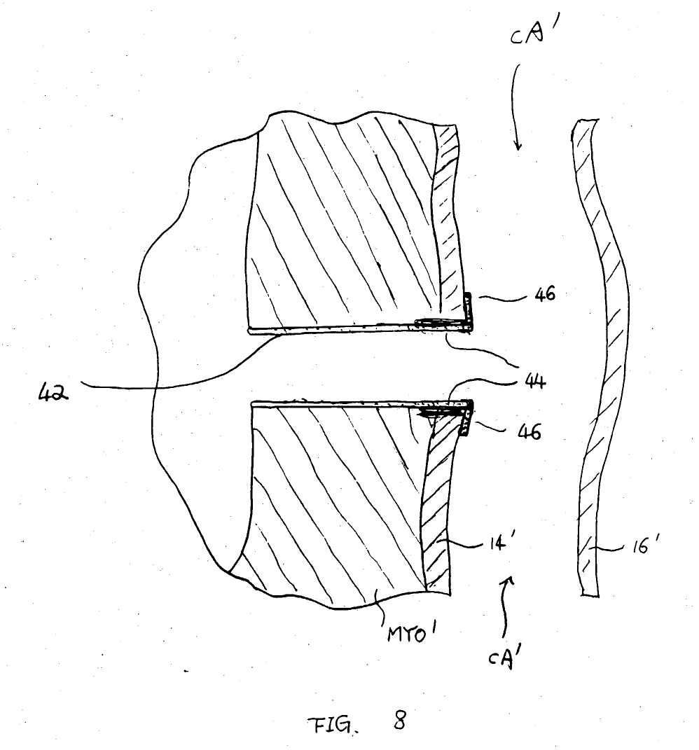

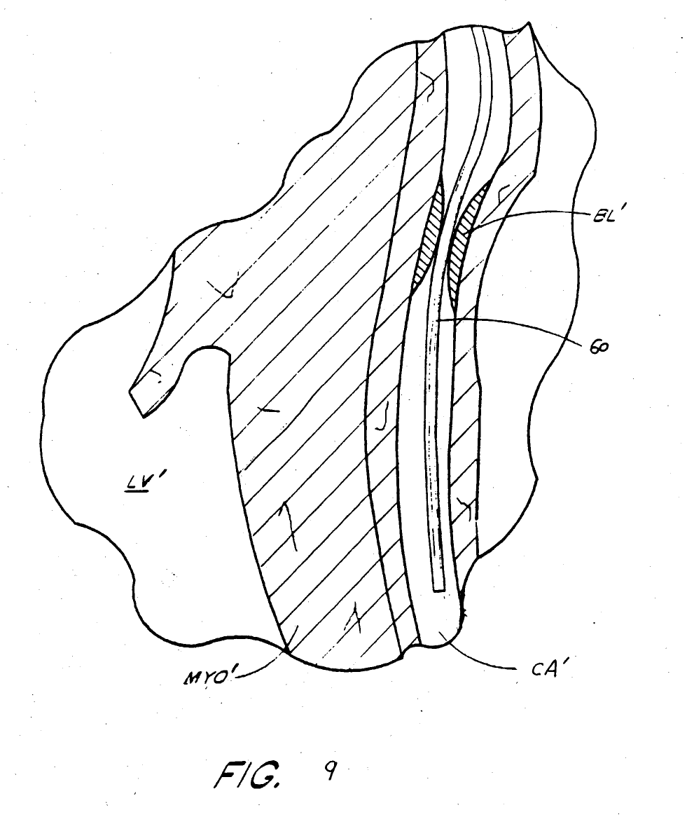

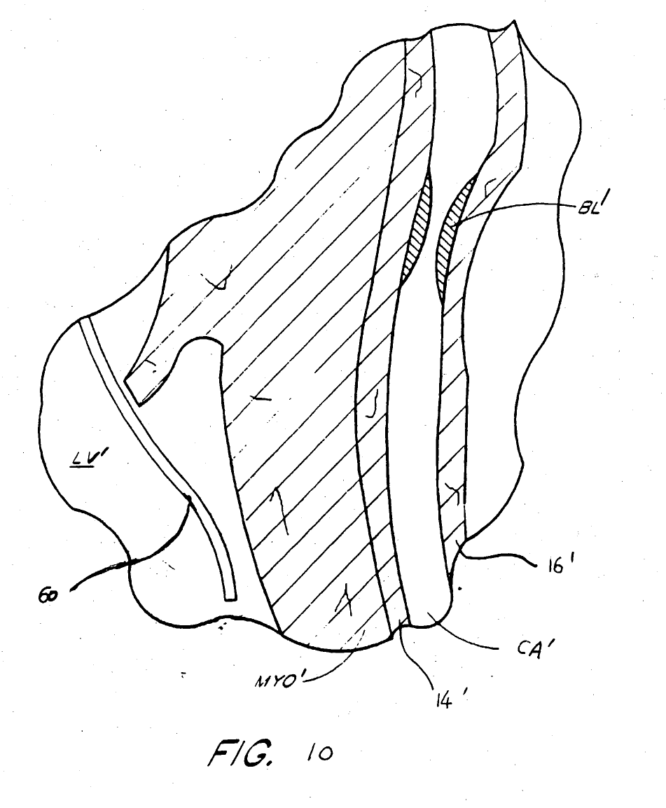

FIG. 7 illustrates an exemplary embodiment in which a collar 20 is welded to an end portion of an implant 42. Although the collar 20 illustrated may be a single-layer coil having a repeatable wave-like form, the collar may be any of the collars discussed above or illustrated in FIGS. 3A-5E, or have other similar suitable structures. The implant 42 also can be any type of implant suitable for insertion into a body part, including, but not limited to, a mesh stent, a non-mesh stent, a coil type stent, or other similar suitable implant, either with or without a covering. As an example, laser welding or other heating methods may form a welding between the collar 20 and the implant 42. In this embodiment, the collar 20 is welded to an end portion of the implant 42 at two peak regions of the coil structure through two laser welding spots 24A and 24B. As shown in FIG. 7, the welding spots 24A and 24B are about 180 degrees apart from each other. Such a bond between a collar and implant may be effective and durable.

In addition to the exemplary welding structure in FIG. 7, the present invention may include alternative welding configurations. For example, the number and location or locations of the welding spots may vary depending on various factors, such as, the desired strength, collar structure, collar material, stent material, size of the collar, size of the stent, and welding methods, for example. In addition, various welding configurations can be adapted in the various types of collars and implants discussed above.

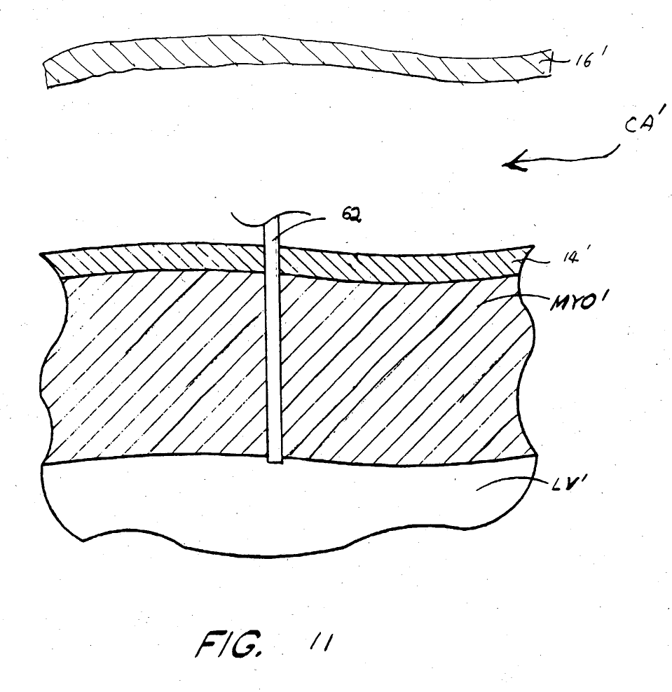

Any of the various types of collars described herein may be combined with an implant that has or does not have an additional attachment mechanism in itself. A collar therefore may be combined with an implant that has its own attachment mechanism, such as, for example, hooks, flares, tabs, barbs, flanges, expandable legs, suture holes, and other structures of similar effects. For an implant that has its own attachment mechanism, the addition of a collar further enhances the anchoring effect to help to prevent the migration of the implant in the heart wall. FIG. 8 shows an exemplary embodiment of an implant 42 that has extensions 46 configured to extend into the coronary artery CA′ or to lie on the surface of a portion of the posterior wall 14′. The collar 44 disposed externally on a portion of the implant 42 proximate the extensions 46 may be any type of collar discussed above. The extensions 46 may be two or more tabs extending from an end of the implant and configured to be bent onto the posterior wall 14′. Alternatively, the extensions may be in the form of barbs, flanges, expandable legs, suture holes, and other similar or suitable structures. The extensions 46 provided by the implant 42 may further assist anchoring the implant 42 within the heart wall MYO′.

Alternatively, the extensions may be portions of the collar 44 that are bent or folded out onto the posterior wall 14′. For example, a portion of any of the collars described above, such as a peak or end region of a collar, may be bent outward and extend into the lumen of the coronary artery CA′ to serve as an additional anchoring mechanism. The extensions (not shown) provided by a collar may further assist anchoring the implant 42 to the heart wall MYO′.

The present invention may include various types of collars and implants and is not limited to the embodiments discussed above. For example, a collar can be any type of structure formed on anywhere along the length or external wall of an implant. The collar may extend from the implant into a body tissue to help anchor the implant in place. In addition, an implant inserted into a body part may have various uses, such as providing flow communication between two body parts, delivering drugs into a body part, or serving as a sensor, controller, or monitoring device. As an example, an implant in the form of a conduit or stent to provide flow communication may be a non-mesh stent, a mesh stent, a coil type stent, or other type of stent. Also, the implant may have a covering on its inside, outside, or both sides. Furthermore, a collar and an implant may be combined anytime, such as before insertion or during insertion into a body part as described below.

The following paragraphs describe exemplary embodiments of methods for inserting an implant and a collar into a body part. The various approaches described below may be used with any of the collars and implants discussed above.

Generally, the combination of a collar and an implant may occur either before insertion or during insertion. The procedure of delivering the collar and implant may vary accordingly.

In a pre-insertion combination, a collar and an implant may be joined together before they are delivered and inserted into a body part. In this instance, a collar may be attached onto an implant before delivery, either in a collapsed form or in a non-collapsed form. A collar also may be welded or sewn to an implant, as discussed above. Also, a welded or sewn combination of a collar and a stent may be crimped to allow ease of delivery. As another example, a collar may also be formed integrally with an implant, such as, for example, by forming an additional structure externally on a portion of the implant when it is made.

As an alternative, for combination occurring during insertion, a collar and implant are provided as two separate units prior to delivery. They may be delivered into a body part, or a location near the body part, either jointly or separately. During insertion, one of them may be inserted first and the other may be inserted second. In one exemplary embodiment, a collar may be inserted into a heart wall first. An implant may then be inserted in place into a heart wall through an opening defined by the inserted collar. As noted above, a portion of the implant also lies in the opening of the collar. The following paragraphs describe the procedures for inserting a collar and implant under both the pre-insertion combination and combination during insertion approaches.

Generally, the installation of a collar and implant may be accomplished by an implant delivery system. An implant delivery system may provide one or more functions, such as providing access to an insertion site or a location near the insertion site, providing a passageway for insertion, delivering a collar and implant into a body, and inserting the collar and implant into the body part, for example. As an exemplary embodiment, one or more catheters may be inserted percutaneously or surgically into a body. The catheter may be inserted into the body with or without a guidewire that guides the entry of the catheter during the insertion process.

In an exemplary embodiment of obtaining access to a heart wall under the percutaneous approach, the catheter may be inserted through the femoral artery and advanced in the patient's vasculature through the aorta. Alternatively, the catheter may obtain access under an open-chest or other surgical approaches. For example, the catheter may be inserted through the anterior wall and posterior wall of a coronary artery and into the heart wall.

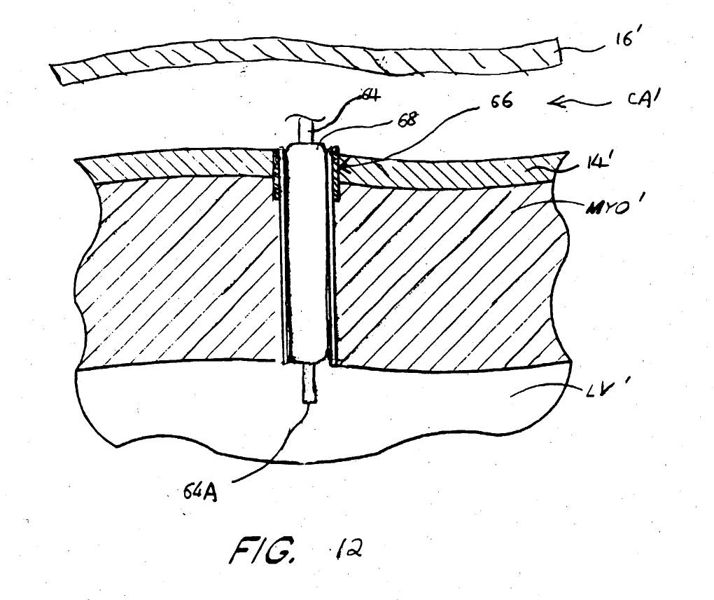

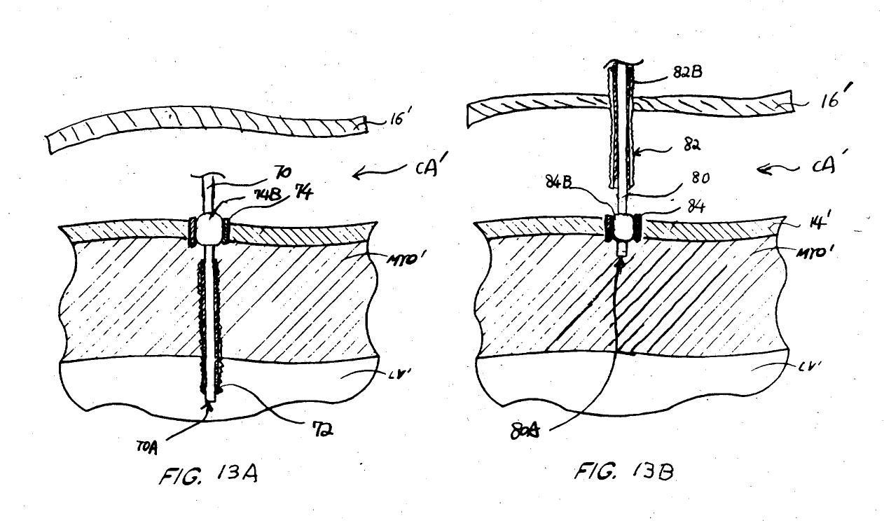

FIGS. 9 and 10 show two exemplary embodiments of providing a catheter with access to the heart wall MYO′ via a percutaneous approach. In FIG. 9, an exemplary delivery catheter or guidewire 60 may be advanced past the blockage BL′ in the coronary artery CA′. In this instance, the catheter or guidewire 60 may have been advanced percutaneously through a femoral artery (not shown) and through aorta (not shown) before advancing through the blockage BL′. FIG. 10 provides an alternative embodiment in which the catheter or guidewire 60 is delivered to a position adjacent the heart wall MYO′ through the left ventricle LV′ after it is advanced percutaneously through aorta (not shown).

As an alternative to the two exemplary percutaneous approaches, a catheter or guidewire may puncture into the heart wall from a lateral location under a surgical approach. For example, a catheter may enter into a heart wall from the right side of the heart wall MYO′ in FIG. 10 by advancing through the anterior wall 16′ (either by puncturing the anterior wall 16′ directly or through an arteriotomy) and the posterior wall 14′ of the coronary artery CA′.

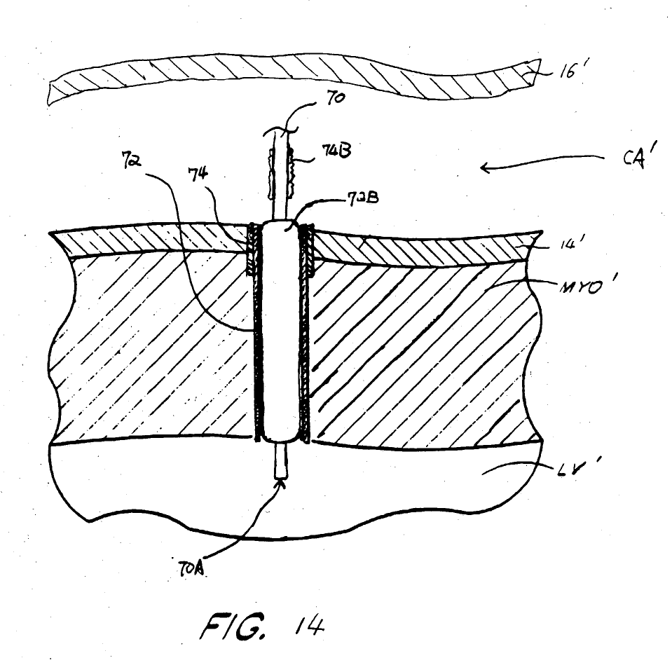

Once a catheter or guidewire obtains access to the heart wall under either a percutaneous or surgical approach, the catheter or guidewire advances into the heart wall. FIG. 11 illustrates an exemplary embodiment with a catheter 62 inserted into the heart wall MYO′. The catheter 62 may be advanced with or without a guidewire and with or without a puncturing needle to assist the positioning and puncturing of the catheter 62. Those skilled in the art would understand various ways to insert the catheter. Although the catheter 62 punctures into the heart wall MYO′ in a direction substantially perpendicular with the lumen of the coronary artery CA′ in this exemplary embodiment, it should be appreciated that the angle of catheter insertion may be adjusted between 0 and 180 degrees with respect to the coronary artery lumen, depending on the desired application and particular conditions of a patient's body.

In this exemplary embodiment, the catheter 62 is an elongate tubular and flexible body having a lumen extending from a proximal end to a distal end. In general, a proximal end is an end that is substantially outside a patient's body for allowing a surgeon or operator to exercise control over a catheter and a distal end is an end that enters into the patient's body and into the heart wall MYO′.

If a guidewire is used, the guidewire is first inserted into a body part, such as a heart wall, before a catheter is inserted into the heart wall. In an exemplary embodiment, a guidewire may be inserted into a heart wall under a percutaneous or surgical approach. The guidewire may extend to a portion of a heart to guide the advancement of a catheter into the heart wall before subsequent insertion of a collar and implant. A guidewire may be equipped with one or more balloons to help to control the guidewire advancement process and also to anchor the front end of the guidewire to a certain body part. For example, a balloon at the front end of a guidewire may expand inside a left ventricle to help to anchor the front end of the guidewire in a heart wall between a coronary vessel and a heart chamber during catheter insertion. After this optional guidewire insertion, a catheter may be inserted by advancing the catheter along the guidewire.

Prior to delivering a collar and implant into the heart wall MYO′ , an optional step may be used to provide a passageway within the heart wall MYO′.

The passageway, if formed, may facilitate the insertion of the collar and implant. In an exemplary embodiment, the catheter 62 in FIG. 11 may have a dilation balloon (not shown) that is advanced to the myocardium or heart wall MYO′, either with or without a guidewire. Once the dilation balloon is in the heart wall MYO′, it expands to push surrounding myocardial tissue outward until a myocardial passageway of desired size is created. The balloon then deflates and the catheter 62 with the dilation balloon may be withdrawn. This process may be repeated with successively larger dilation balloons, if necessary to form a passageway of desired size. Other dilation mechanisms, such as ablation tools or a series of dilating catheters, also may be employed to form such a passageway and are considered within the scope of their invention.

Once the catheter obtains access to the heart wall under either a percutaneous or surgical approach, the catheter advances into the heart wall. In one exemplary embodiment, a catheter 64 advances into the heart wall MYO′, delivers a collar and implant, and inserts the collar and implant in the heart wall MYO′ as shown in FIG. 12. As discussed above, the catheter 64 may advance into the heart wall MYO′ with or without the optional step of providing a passageway. In one exemplary embodiment, the catheter 64 may carry both the collar and implant at a location near its distal or inserting end 64A.

FIG. 12 shows an exemplary embodiment in which the collar and implant are delivered as one combined unit of a collared implant 66. In this example, the collar is joined to the implant before delivery. Alternatively, a collar and implant may be joined during insertion, the process of which will be described below.

A collar may assist a surgeon or operator to determine the location of the collared implant 66 during the insertion process. In one exemplary embodiment, the collar is joined to an external portion of the implant and the implant is fitted externally on a balloon 68 of the catheter 64. Referring to FIG. 12, the collar therefore provides an external, radial extension from the external wall of the implant, and also from the catheter 64. Due to such extension, once the collar engages with the posterior wall 14′ of the coronary artery CA′ during insertion, the advancement of the catheter 64 becomes more difficult and requires more force to push the catheter 64 into the posterior wall 14′ and the heart wall MYO′. Based on the increased resistance against advancement, the surgeon or operator may be able to determine the relative location of the collared implant 66 to the heart wall MYO′ to assist in proper positioning of the collared implant 66.

The insertion of a collar and implant may involve expanding the collar and implant, depending on the types of the collar and implant delivered. For a non-self-expandable collared implant, the catheter 64 may include an inflation balloon 68, as shown in the exemplary embodiment of FIG. 12. The collared implant 66 is first delivered to a desired location in the heart wall MYO′ . The balloon 68 then inflates to expand the collared implant 66 and to push outward against surrounding myocardial tissue. After the desired disposition of the collared implant 66, the balloon 68 deflates and the catheter 64 may be withdrawn to leave the collared implant 66 in place. The collared implant 66 in the form of a stent or conduit, therefore, may provide a flow communication between two body parts, such as between the left ventricle LV′ and coronary artery CA′.

For a self-expandable collared implant, a retaining sheath, which may be in the form of a catheter, for example, may be used to restrain the collared implant from expanding before its insertion into a body part. The sheath and the collared implant are first delivered to a desired location. The retaining sheath may then be removed or retracted to allow the collared implant to self-expand. The expanded collared implant in the form of a stent or conduit may provide a flow communication between two body parts, such as between the left ventricle LV′ and coronary artery CA′ in FIG. 12.

Although the embodiment of FIG. 12 is illustrated with the collar placed at an end portion of the implant proximate the coronary artery CA′, the collar may be placed anywhere along the length or the external wall of the implant. In one embodiment, the location of the collar relative to the implant may be adjusted when the collar is joined with the implant prior to delivery and insertion.

Aside from the pre-insertion combination approach discussed above, an implant and a collar may be delivered and/or inserted sequentially under the approach of combination during insertion. As described above, a catheter first obtains access to a heart wall under either a percutaneous or surgical approach, and it then advances into the heart wall, either with or without the use of a guidewire. A single catheter that is advanced to the heart wall may carry both a collar and implant. Alternatively, two delivery catheters may be used to carry a collar and implant separately and may be advanced to the heart wall sequentially. In addition, an optional step may be used to provide a passageway within the heart wall prior to catheter insertion.

FIG. 13A shows an exemplary embodiment in which an implant delivery catheter 70 carries an implant 72 near a distal end 70A of the catheter 70, and also carries a collar 74 proximal the implant 72. Both the implant 72 and collar 74 may be carried by the catheter 70 in a crimped or collapsed shape. Both the implant 72 and collar 74 may also have inflation balloons underneath them for expanding them. In the exemplary embodiment shown in FIG. 13A, a first balloon 74B of the catheter 70 first inflates to expand the collar 74 when this balloon-expanded collar 74 is advanced to a designated position, such as, for example, to an end portion of a passageway proximate the coronary artery CA′.

Upon the desired disposition of the collar 74, the first balloon 74B deflates and the catheter 70 is retracted to a location for inserting the implant 72, as shown in FIG. 14. The catheter 70 and implant 72 may be retracted through a hollow space within the expanded collar 74. The implant 72 is then carried to a designated position, such as a place where the implant 72 is substantially within the heart wall MYO′, or where one end of the implant 72 is substantially flush with the lumen of the coronary artery CA′. And a second balloon 72B of the catheter 70 then inflates to expand the implant 72. Upon the desired disposition of the implant 72, the second balloon 72B deflates and the catheter 70 may be completely withdrawn from the patient's body. The implant 72 in the form of a stent or conduit, therefore, may start to provide flow communication between the left ventricle LV′ and the coronary artery CA′ after the catheter 70 is removed.

FIG. 13B shows another exemplary embodiment in which a catheter 80 carries a collar 84 near a distal end 80A of the catheter 80, and carries an implant 82 proximal the collar 84. In this example, the catheter 80 may be inserted with a surgical approach by puncturing through the anterior wall 16′ of the coronary artery CA′. The catheter 80 may carry both the implant 82 and collar 84 in a crimped or collapsed shape outside the catheter 80. Furthermore, both the implant 82 and collar 84 may have inflation balloons underneath them for expanding them. In an exemplary embodiment, the collar 84 is first inserted in a designated position. A first balloon 84B of the catheter 80 then inflates to expand the balloon-expanded collar 84. Upon the desired disposition of the collar 84, the first balloon 84B deflates and the catheter 80 is advanced to a location for inserting the implant 82.

Similar to the example in FIG. 13A, the catheter 80 and the implant 82 in FIG. 13B may advance through an opening defined by the collar 84. The implant 82 is then advanced to a designated place, such as a position where the implant 82 is substantially within the heart wall MYO′, or where one end of the implant 82 is substantially flush with the lumen of the coronary artery CA′. A second balloon 82B of the catheter 80 then inflates to expand the implant 82. The configuration of the expanded collar 84 and implant 82 is similar to that of the expanded collar 74 and implant 72 in FIG. 14. Upon the desired disposition of the implant 82, the second balloon 82B deflates and the catheter 80 may be completely withdrawn from the patient's body. The implant, if in the form of a stent or conduit, may start to provide flow communication between the left ventricle LV′ and the coronary artery CA′ after the catheter 80 is removed.

Although the embodiments in FIGS. 13A-14 are illustrated with balloon-expanded collars and implants, similar approaches may be applied for installing self-expandable collars and implants. In those instances, retaining sheaths may be used to keep a collar or implant in a collapsed shape prior to its insertion. Once the collar or implant is positioned at a designated location, a corresponding sheath may be removed or retracted to allow the collar or implant to expand in a body part. Furthermore, although the embodiments in FIGS. 13A-14 are illustrated with a collar placed at an end portion of an implant proximate to the coronary artery CA′, the collar may be placed anywhere along the length or external wall of the implant by adjusting the location of inserting the collar. Under these approaches of combining the collar and implant upon insertion, the location of a collar relative to an implant and a heart wall may be adjusted freely during the insertion process. Alternatively, under the approach of inserting a combined unit of collared implant, the location of the collar relative to the implant is usually adjusted prior to delivery and insertion.

As noted above, various methods and delivery tools may be used to deliver and insert a collar and an implant into different body parts. Skilled artisans may use any embodiments, modifications and variations thereof, and other conventional techniques to deliver and insert any collar and implant of the invention. In other words, skilled artisans may use delivery or insertion techniques known in the art to deliver or insert the device of the invention. The exemplary embodiments above, therefore, are not intended to limit the use of their modifications, variations, other known techniques, and techniques that skilled artisans learn from practicing the invention.

Further, although the exemplary embodiments described above have been discussed in the context of implanting a collared stent in a heart wall between the left ventricle and a coronary artery, the various devices and methods disclosed are not limited to that application. Rather, they can be employed for connecting any heart chamber to any coronary vessel, for example a vein, or for connecting two coronary vessels. In addition, as noted above, the collared implants may be used in any body part or body tissue for delivery of drugs, providing flow communication between two body parts, or for sensing, diagnosing, or monitoring body parts.

It will be apparent to those skilled in the art that various modifications and variations can be made in the exemplary devices and methods described above and in the construction of those devices and methods. Other embodiments of the invention will be apparent to those skilled in the art from consideration of the specification and practice of the invention disclosed herein. It is intended that the specification and examples be considered as exemplary only.

What is claimed is:

1. A device for treating a heart, comprising:
an implant configured to be positioned in a heart wall between a coronary vessel and a chamber of the heart; and
a collar configured to be disposed externally on a portion of the implant such that the collar extends from the implant so as to contact the heart wall for anchoring the implant in position in the heart wall.

2. The device of claim 1, wherein the collar is expandable.

3. The device of claim 2, wherein the collar is elastic.

4. The device of claim 2, wherein the collar is collapsible.

5. The device of claim 1, wherein the collar is a mesh collar.

6. The device of claim 1, wherein the collar comprises a coil.

7. The device of claim 6, wherein the coil wraps around the external portion of the implant in a wave-like pattern.

8. The device of claim 7, wherein the wave-like pattern comprises a sinusoidal wave-like pattern.

9. The device of claim 1, wherein the collar comprises an upper coil that wraps around the implant in a first wave-like pattern and a lower coil that wraps around the implant in a second wave-like pattern.

10. The device of claim 9, wherein the first wave-like pattern comprises a first sinusoidal wave-like pattern and the second wave-like pattern comprises a second sinusoidal wave-like pattern.

11. The device of claim 9, wherein the collar comprises at least one interconnection between the upper coil and the lower coil.

12. The device of claim 11, wherein the interconnection comprises at least one flexible joint.

13. The device of claim 12, wherein the flexible joint is configured to permit relative movement between the upper coil and the lower coil.

14. The device of claim 12, wherein the flexible joint is configured to permit at least one of extension, contraction, bending, and twisting of the collar.

15. The device of claim 1, further comprising a connection for connecting the collar to the implant, wherein the connection is chosen from a welding connection and a sewing connection.

16. The device of claim 1, wherein the collar is configured to be disposed on an end portion of the implant.

17. The device of claim 16, wherein the collar is configured to be positioned proximate the coronary vessel when the implant is positioned in the heart wall.

18. The device of claim 1, wherein the collar is configured to have portions of the collar substantially surrounded by heart tissue when the collar is implanted in the heart wall.

19. The device of claim 1, wherein the collar includes extensions configured to extend into a lumen of the coronary vessel.

20. The device of claim 1, wherein the collar comprises a biocompatible material.

21. The device of claim 1, wherein the collar comprises a material chosen from stainless steel, nickel alloys, titanium alloys, nickel-titanium alloys, cobalt-based alloys, titanium, and tantalum.

22. The device of claim 1, wherein the collar comprises a biocompatible polymer material.

23. The device of claim 22, wherein the biocompatible polymer material is chosen from polytetrafluoroethylenes, polyetheretherketones, polyesters, polyurethanes, polyamides, and expanded polytetrafluoroethylenes.

24. The device of claim 1, wherein the collar is self-expandable.

25. The device of claim 1, wherein the collar is in the form of an annulus.

26. The device of claim 1, wherein the implant comprises an anchoring mechanism configured to engage with a portion of the coronary vessel.

27. The device of claim 26, wherein the anchoring mechanism comprises at least one mechanism chosen from hooks, flares, tabs, barbs, flanges, expandable legs, and suture holes.

28. The device of claim 1, wherein the implant is configured to provide flow communication between the heart chamber and the coronary vessel when the implant is positioned in the heart wall.

29. The device of claim 1, wherein the implant is configured to be positioned in the heart wall between a left ventricle and a coronary artery.

30. The device of claim 1, wherein the implant comprises a conduit.

31. The device of claim 30, wherein the conduit is a mesh conduit.

32. The device of claim 31, wherein at least a portion of the mesh conduit is covered with a polymer material.

33. The device of claim 30, wherein the conduit is a stent.

34. The device of claim 30, wherein the conduit comprises a coil structure.

35. The device of claim 30, further comprising a covering over at least a portion of the conduit.

36. The device of claim 35, wherein the covering is made of an expanded polytetrafluoroethylene.

37. The device of claim 1, wherein the collar is a coil structure formed by one of laser cutting and an electrochemical method.

38. A method of treating a heart, comprising:
providing a collar and an implant, the collar being configured to be disposed externally on a portion of the implant;
delivering the collar and the implant to a location proximate to a heart wall; and
inserting the collar and the implant into the heart wall between a coronary vessel and a heart chamber, wherein the collar is configured to extend from the implant so as to contact the heart wall for anchoring the implant in position in the heart wall.

39. The method of claim 38, further comprising advancing a guidewire to the heart wall between the coronary vessel and the heart chamber before delivering the collar and the implant to the location proximate to the heart wall.

40. The method of claim 38, further comprising forming a passageway in the heart wall between the coronary vessel and the heart chamber and inserting the collar and the implant into the passageway.

41. The method of claim 38, further comprising flowing blood through the implant between the heart chamber and the coronary vessel after inserting the collar and the implant.

42. The method of claim 38, wherein providing the collar and the implant comprises providing a connection between the collar and the implant, wherein the connection is chosen from a welding connection and a sewing connection.

43. The method of claim 38, wherein providing the collar comprises providing an expandable collar.

44. The method of claim 43, wherein providing the collar comprises providing an elastic collar.

45. The method of claim 43, wherein providing the collar comprises providing a collapsible collar.

46. The method of claim 38, wherein providing the collar comprises providing a mesh collar.

47. The method of claim 38, wherein providing the collar comprises providing a collar comprising a coil.

48. The method of claim 47, wherein the coil wraps around the external portion of the implant in a wave-like pattern.

49. The method of claim 48, wherein the wave-like pattern comprises a sinusoidal wave-like pattern.

50. The method of claim 38, wherein providing the collar comprises providing a collar that comprises an upper coil that wraps around the implant in a first wave-like pattern and a lower coil that wraps around the implant in a second wave-like pattern.

51. The method of claim 50, wherein the first wave-like pattern comprises a first sinusoidal wave-like pattern and the second wave-like pattern comprises a second sinusoidal wave-like pattern.

52. The method of claim 50, wherein the collar comprises at least one interconnection between the upper coil and the lower coil.

53. The method of claim 52, wherein the interconnection comprises at least one flexible joint.

54. The method of claim 53, wherein the flexible joint is configured to permit relative movement between the upper coil and the lower coil.

55. The method of claim 53, wherein the flexible joint is configured to permit at least one of at least one of extension, contraction, bending, and twisting of the collar.

56. The method of claim 38, wherein providing the collar comprises providing a collar that comprises a biocompatible material.

57. The method of claim 38, wherein providing the collar comprises providing a collar that comprises a material chosen from stainless steel, nickel alloys, titanium alloys, nickel-titanium alloys, cobalt-based alloys, titanium, and tantalum.

58. The method of claim 38, wherein providing the collar comprises providing a collar that comprises a biocompatible polymer material.

59. The method of claim 58, wherein the biocompatible polymer material is chosen from polytetrafluoroethylenes, polyetheretherketones, polyesters, polyurethanes, polyamides, and expanded polytetrafluoroethylenes.

60. The method of claim 38, wherein providing the implant comprises providing a conduit.

61. The method of claim 38, wherein delivering the collar and the implant comprises delivering the collar and the implant to the location proximate to the heart wall via a catheter.

62. The method of claim 38, wherein delivering the collar and the implant comprises:
delivering the implant to the location proximate to the heart wall via a first catheter; and
delivering the collar to the location proximate to the heart wall via a second catheter.

63. The method of claim 38, wherein inserting the collar and the implant comprises inserting the implant with the collar attached to the external portion of the implant.

64. The method of claim 38, wherein inserting the collar and the implant comprises:
inserting the collar into the heart wall between the coronary vessel and the heart chamber; and
inserting the implant into the heart wall through an opening defined by the collar.

65. The method of claim 38, wherein inserting the collar and the implant comprises inserting the collar and the implant such that the collar is positioned on an end portion of the implant proximate the coronary vessel.

66. The method of claim 38, wherein inserting the implant comprises implanting the implant in a position within the heart wall between a left ventricle and a coronary artery.

67. The method of claim 38, wherein inserting the collar and the implant comprises expanding the collar and the implant.

68. The method of claim 67, wherein expanding the collar and the implant includes expanding the collar and implant via a balloon.

69. The method of claim 67, wherein the collar and the implant are self-expandable.
